(19) United States
(12) Patent Application Publication
(10) Pub. No.: US 2014/0128585 A1
(43) Pub. Date: May 8, 2014

(54) FRUCTOSE ABSORPTION INHIBITOR

(71) Applicants: Keiichiro Sugimoto, Osaka (JP); Hiroto Nakayama, Osaka (JP); Kazuya Nakagawa, Osaka (JP); Syuichi Hayashi, Osaka (JP)

(72) Inventors: Keiichiro Sugimoto, Osaka (JP); Hiroto Nakayama, Osaka (JP); Kazuya Nakagawa, Osaka (JP); Syuichi Hayashi, Osaka (JP)

(73) Assignee: Nagaoka Perfumery Co., Ltd., Osaka (JP)

(21) Appl. No.: 14/130,104

(22) PCT Filed: Jul. 6, 2012
(86) PCT No.: PCT/JP2012/067346

§ 371 Date: Dec. 30, 2013

(30) Foreign Application Priority Data
Jul. 7, 2011 (JP) 2011-151059

(51) Int. Cl. A61K 31/7024; A23K 1/16; A23L 1/30

(52) U.S. Cl. CPC A61K 31/7024; A23L 1/3002; A23K 1/1612
USPC 536/18.1

(57) ABSTRACT

A fructose absorption inhibitor according to the present invention comprises a hydrolyzable tannin as an active ingredient. The hydrolyzable tannin preferably has a form composed of a gallic acid derivative and/or an ellagic acid derivative bound to a hydroxy group in glucose via an ester bond, and includes ellagitannin, gallotannin and so on.

### TECHNICAL FIELD

The present invention relates to a fructose absorption inhibitor which inhibits the process of absorbing fructose contained in foods and drinks into the body through the small intestine to prevent and improve lifestyle-related diseases such as obesity, fatty liver, diabetes and the like caused by fructose intake.

### BACKGROUND ART

Obesity is a state in which fat accumulates excessively in the body. One of the causes of fat accumulation in the body is excess intake of saccharides (carbohydrates). In general, when saccharides contained in foods and drinks are taken into the body, saccharides are digested by digestive enzymes, mainly converted into monosaccharides and absorbed into the body through the intestinal tracts.

One kind of monosaccharides, glucose, is digested and absorbed, and then metabolized by group of enzymes called the glycolytic pathway. Since the metabolism is regulated at the step with phosphofructokinase among these enzymes, the fat synthetic pathway is not immediately activated even if a large amount of glucose is taken.

On the other hand, another kind of the monosaccharides, fructose, is metabolized through a pathway bypassing the step with phosphofructokinase unlike glucose. Therefore, if fructose is taken in a large amount, fructose rapidly proceeds to the fat synthetic pathway in the liver and generated fat accumulates in adipose tissue. Accordingly, the usual intake does not cause problems on health and safety, but excess intake of fructose increases the risk of causing pathological conditions such as obesity.

In addition, fructose has strong sweetness among monosaccharides and has such a property that its sweetness is enhanced as the temperature decreases. Therefore, fructose is widely used as a sweetener for processed foods such as chilled sweets and soft drinks containing high-fructose corn syrup (HFCS) and the consumption has been rapidly increasing in recent years.

It is well known that the excessive intake of such soft drinks containing high-fructose corn syrup has become a social problem in each country. Also in Japan, mainly among young males, cases that are called “PET bottle syndrome” or “soft drink ketosis” have been increasing, wherein daily intake of 1 liter or more of soft drink causes ketosis or ketoacidosis. Further, diabetes patients have increased concentration of fructose in the blood or increased excretion of fructose in the urine. Especially, it has been reported that the high postprandial concentration of fructose in the blood correlates with diabetic retinopathy (Non-patent Document 1), which reports excess intake of fructose can be a cause of diabetic complications.

Under such social background, WHO (World Health Organization) recommends that the intake of saccharides added as sweeteners should not exceed 10% of the total energy intake (Non-patent Document 2). For example, in the United States, ADA (American Diabetes Association) warns against use of fructose as an alternative to sucrose in a guideline for medical care for diabetes. Also in Japan, Ministry of Health, Labour and Welfare raises an alarm over excess intake of fructose in “The Dietary Reference Intakes for Japanese”, 2010 edition, developed by Ministry of Health, Labour and Welfare.

Excess intake of fructose can be a factor causing various diseases other than those mentioned above. That includes enhancement of oxidative stress in vivo (Non-patent Document 2), glycation of protein (Non-patent Documents 3 and 4), calcium deposits in the kidney (Non-patent Document 5), hyperuricemia (Non-patent Document 6), initiation of insulin resistance (Non-patent Document 7), initiation of cardiovascular kidney diseases (Non-patent Document 8), non-alcoholic fatty liver disease (NAFLD) (Non-patent Document 9) and the like.

Glycation of protein means generation of AGE (Advanced Glycation End-product) through reactions between saccharides and proteins in vivo. In the process of the reactions, cellular aging and denaturation of protein occur. Generated AGEs also react with surrounding proteins and the like and promote denaturation of body tissues and the like. Since AGE is involved in aging of capillary blood vessels and the like, AGE is regarded as one of the causes of cataract, decrease in kidney function and the like. In particular, AGE is considered to be strongly related to the onset and aggravation of complications caused by diabetes progression. Fructose has a strong reducing character. Therefore, there have been many reports that fructose has much greater glycation power than glucose. Methylglyoxal, a by-product in the process of fructose metabolism, is also problematic as a precursor of AGE.

Incidentally, one kind of the saccharides, sugar (sucrose), is decomposed into the glucose and fructose by amylolytic digestive enzymes. When a large amount of sugar is taken, rapid absorption of glucose leads to surge in the blood glucose level along with immediate secretion of insulin. Insulin helps to promote the pathway to convert fructose and glucose into lipid, glycogen synthesis and glucose intake by adipocytes. Therefore, medical importance is also placed on sugar as a substance causing obesity and the like.

However, sugar is the best as a sweetener considering taste, so a large amount of sugar is consumed for juice, confectionary, cooking and the like.

Accordingly, in cases where a large amount of sugar is taken, if the absorption of glucose and fructose generated by digestion of sugar into the body can be inhibited, the effect of reducing the total calorie intake can be expected. However, glucose is the most important monosaccharide for mammals biochemically and is the major energy source for various tissues. In particular, the brain usually uses glucose as a sole energy source. Therefore, it is problematic to strongly inhibit absorption of glucose in view of safety.

On the other hand, as mentioned above, few roles are confirmed about fructose except for a role as a calorie source. Then, less importance is placed on fructose than glucose nutritionally. Therefore, it can be considered that as a preventive measure against obesity and the like in cases where saccharides are taken excessively, the best way is to specifically inhibit the process of absorbing fructose through the intestinal tracts into the body.

As conventional knowledge, concerning substances which specifically inhibit absorption of fructose into the body, an extract of eucalyptus leaves (Patent Document 1), several kinds of natural extracts (Patent Document 2), and glyco-1,3-oxazolidin-2-ones and analogs thereof that have been synthesized as analogs of fructose or sorbitol (Non-Patent Document 10) have been reported.

However, these natural extracts have a weak inhibitory activity and need to be used in a large amount. In addition, there are problems that it is difficult to maintain the quality constant since natural extracts are a mixture and that natural extracts are hard to be added to foods, drinks, animal feed or the like due to their distinctive tastes. Synthetic substances cannot be used for foods and drinks, and safety needs to be strictly verified if they are used for drugs. So it is very difficult to put them to practical use.

### PRIOR ART DOCUMENTS

### Patent Documents

Patent Document 1: Japanese Unexamined Patent Application Publication 2003-160504
Patent Document 2: Japanese Unexamined Patent Application Publication 2009-184992

### Non-Patent Documents

Non-Patent Document 1: T. Kawasaki et al., Metabolism, 53, 583-588 (2004)
Non-Patent Document 2: A. Cavarape et al., J. Endocrinol. Invest., 24, 838-845 (2001)
Non-Patent Document 3: Takahiro Kawasaki et al., Diabetes, 48, 419-421 (2005)
Non-Patent Document 4: C. G. Schalkwijk et al., Diabetes Metab. Res. Rev., 20, 369-382 (2004)
Non-Patent Document 5: Takatoshi Esashi, SHOKUHIN EISEIGAKU ZASSHI, 35, 409-412 (1994)
Non-Patent Document 6: X. Gao et al., Hypertension, 50, 306-312 (2007)
Non-Patent Document 7: S. S. Elliott et al., Am. J. Clin. Nutr., 76, 911-922 (2002)
Non-Patent Document 8: R. J. Johnson et al., Am. J. Clin. Nutr., 86, 899-906 (2007)
Non-Patent Document 9: T. Kawasaki et al., J. Nutr., 139, 2067-2071 (2009)
Non-Patent Document 10: J. Girniene et al., Carbohyd. Res., 338 (2003)

### SUMMARY OF THE INVENTION

### Problems to be Solved by the Invention

Accordingly, it is desirable to provide a fructose absorption inhibitor which has rich history of use in foods with high safety and exerts a sufficient effect with a small dosage for adding it to various foods, drinks and animal feed and for using it as a drug. Further, stable and inexpensive availability in a large amount of such a fructose absorption inhibitor is industrially very useful.

An object of the present invention is to provide a fructose absorption inhibitor which is derived from natural products, able to specifically inhibit absorption of fructose into the body, safe, applicable to various foods, drugs and animal feed, and easy to take.

### Means for Solving the Problems

The present inventors intensively studied to solve the problems. As a result, they found that a hydrolyzable tannin inhibits fructose absorption through the intestinal tracts very strongly, leading to the present invention. The hydrolyzable tannin has an ester bond composed of a hydroxyl group in a monosaccharide such as glucose and an ellagic acid derivative and/or a gallic acid derivative.

That is, a fructose absorption inhibitor of the present invention includes the following configurations.

(1) A fructose absorption inhibitor containing a hydrolyzable tannin as an active component.

(2) The fructose absorption inhibitor as set forth in (1), wherein the hydrolyzable tannin is a compound having an ester bond composed of a hydroxy group in glucose and a gallic acid derivative and/or an ellagic acid derivative.

(3) The fructose absorption inhibitor as set forth in (1) or (2), wherein the hydrolyzable tannin is ellagitannin having an ester bond composed of hydroxy groups at the 2- and 3-positions or the 4- and 6-positions or at the both positions in glucose and at least one hexahydroxydiphenoyl group or valoneoyl group.

(4) The fructose absorption inhibitor as set forth in (1) or (2), wherein the hydrolyzable tannin is gallotannin having an ester bond composed of at least three hydroxy groups in glucose and gallic acids.

### Effect of the Invention

The fructose absorption inhibitor of the present invention has a high fructose absorption inhibitory effect in the intestinal tracts. Therefore, the fructose absorption inhibitor is effective to prevent, improve and treat obesity and various diseases caused by excess intake of fructose. Moreover, the hydrolyzable tannin used in the present invention is often contained in natural products used for foods and the like, which also ensures high safety. Further, the hydrolyzable tannins used in the present invention are easy to be taken or administered, and the effect can be expected with a small amount of usage as well. Therefore, the fructose absorption inhibitor of the present invention can be contained in various foods, drugs, animal feed and the like. Then various foods, drugs, animal feed and the like suitable for intake or administration can be produced.

### MODE FOR CARRYING OUT THE INVENTION

The fructose absorption inhibitor of the present invention contains a hydrolyzable tannin as an active component. The fructose absorption inhibitor may also be contained in foods, drinks, animal feed, quasi drugs or drugs.

A hydrolyzable tannin is a kind of polyphenols and has an ester bond composed of a polyol such as glucose and a gallic acid derivative and/or an ellagic acid derivative. In the present invention, the hydrolyzable tannin preferably has an ester bond composed of glucose and a gallic acid derivative and/or an ellagic acid derivative and the glucose may be ring-opened.

The gallic acid derivatives include, as represented by the following general formula (1), compounds having the gallic acid structure as a basic skeleton with a hydroxy group in a gallic acid substituted with an alkyl group, an acyl group or the like.

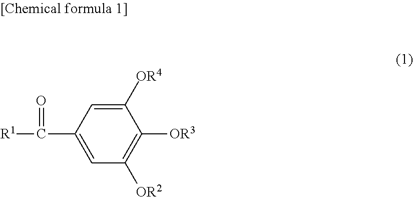

[wherein R¹ represents a hydroxy group or an alkoxy group and R², R³ and R⁴, which are the same or different, each represent a hydrogen atom, an alkyl group or an acyl group.]

The gallic acid derivative may be an oligomer such as a dimer or a trimer in which several structures represented by the general formula (1) are bound to each other via ether bond or ester bond; compounds in which a structure represented by the general formula (1) is bound to one or more ellagic acid derivatives via an ether bond or ester bond; and the like.

Examples of the ellagic acid derivatives include compounds having the ellagic acid structure as a basic skeleton with its hydroxy group substituted with an alkyl group, an acyl group or the like. For example, they are represented by the following general formula (2). In addition, a compound having a hexahydroxydiphenoyl (hereinafter referred to as “HHDP” for short) group derived from an oxide of an ellagic acid and a compound having a valoneoyl group in which a gallic acid is added to an HHDP group are also included in ellagic acid derivatives.

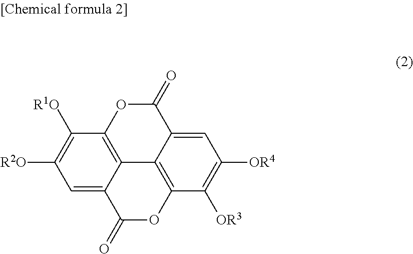

[wherein R¹, R², R³ and R⁴, which are the same or different, each represent a hydrogen atom, an alkyl group or an acyl group.]

The ellagic acid derivative may be compounds in which a structure represented by the general formula (2) is bound to one or more gallic acid derivatives represented by the general formula (1) via an ether bond or ester bond; an oligomer such as a dimer or a trimer in which several structures represented by the general formula (2) are bound to each other via ether bond or ester bond; and the like.

As the hydrolyzable tannin, both of ellagitannin and gallotannin may be preferably used.

Ellagitannin refers to compound groups having an ester bond composed of a hydroxy group in a polyhydric alcohol such as glucose and an HHDP group derived from an oxide of an ellagic acid, and an ellagic acid and a polyhydric alcohol are generated by hydrolysis.

Gallotannin refers to compound groups having an ester bond composed of a hydroxy group in a polyhydric alcohol and a gallic acid, and a gallic acid and a polyhydric alcohol are generated by hydrolysis.

More preferably, the hydrolyzable tannins include, for example, ellagitannin having an ester bond composed of hydroxy groups at the 2- and 3-positions or the 4- and 6-positions or at the both positions in glucose and at least one HHDP group or valoneoyl group; gallotannin having an ester bond composed of at least three hydroxy groups in glucose and gallic acids; and the like.

As used herein, a valoneoyl group is a group having an ether bond composed of an HHDP group and a galloyl group illustrated in the following structure (3). Examples of the ellagitannin also include many compounds having an ester bond composed of other hydroxy group in a polyhydric alcohol and a gallic acid.

Specific examples of the hydrolyzable tannin include, as illustrated in the following structures (4) to (8), Tellimagrandin I and II, Strictinin, Casuarictin, 1,3-di-O-galloyl-4,6-HHDP-β-D-glucose, Oenothein B, Eugeniflorin D₂, 1,2,3-trigalloyl glucose, 1,2,3,6-tetragalloyl glucose, 1,2,3,4,6-pentagalloyl glucose and the like. Among these, the hydrolyzable tannin is desirably at least one selected from the group consisting of Tellimagrandin I and II, 1,3-di-O-galloyl-4,6-HHDP-β-D-glucose, Oenothein B and Eugeniflorin D₂, or at least one selected from the group consisting of 1,2,3-trigalloyl glucose, 1,2,3,6-tetragalloyl glucose and 1,2,3,4,6-pentagalloyl glucose.

These are compounds illustrated in the following structural formulae (4) to (8). Note that G in the following structural formulae (4) to (8) represents the following structure (3).

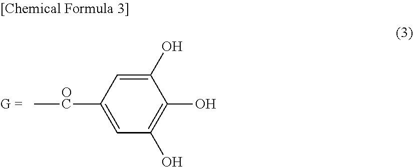

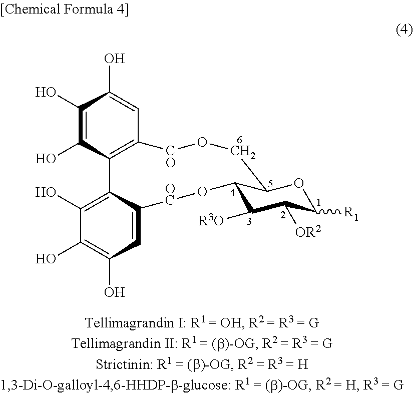

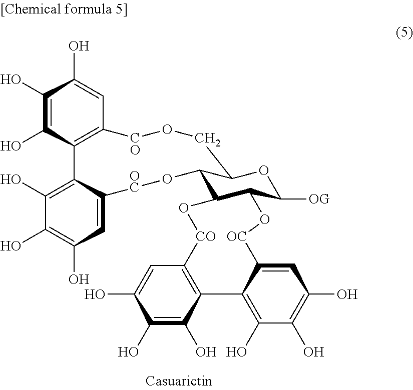

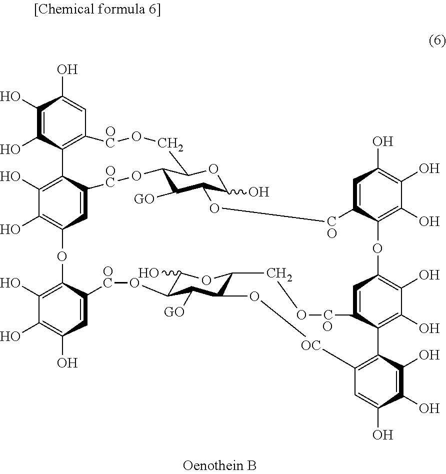

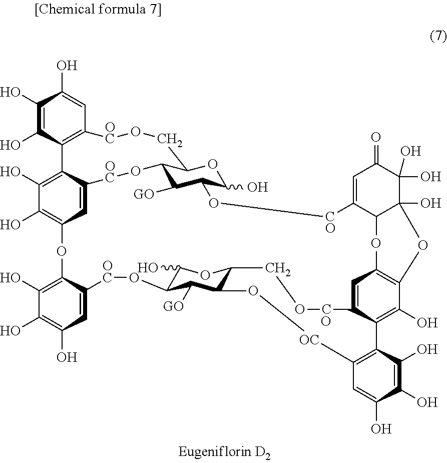

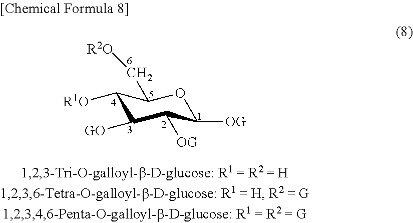

Examples of the raw materials for the hydrolyzable tannin include, but are not particularly limited to, Archichlamydeae belonging to Angiospermae Dicotyledoneae (Engler system) containing a hydrolyzable tannin, a tannic acid and the like.

A method for producing the hydrolyzable tannin having fructose absorption inhibitory activity from the plants is not particularly limited and the hydrolyzable tannin may be produced by commonly used methods. In addition, when the hydrolyzable tannin is obtained by extraction, there are no specific limitations to the extraction conditions. For example, various parts of the plants (whole plant, flower, calyx, seed, fruit, leaf, branch, bark, root bark, rhizome, root and the like) may be squeezed or extracted by a solvent as they are or after cut, ground or pulverized to obtain an extract of the hydrolyzable tannin.

Examples of the plants include Myrtaceae, Rosaceae, Casuarinaceae, Fagaceae, Theaceae, Onagraceae, Lythraceae, Trapaceae, Punicaceae, Melastomataceae, Combretaceae, Lecythidiaceae and the like. Since these plants contain the hydrolyzable tannin in a large amount, the hydrolyzable tannin may be efficiently obtained by using these plants as a raw material. Among these, Myrtaceae plants are preferable. Further, plants belonging to Eucalyptus, Syzygium, Pimenta and Melaleuca are often used for foods, spices, flavors and the like. Therefore, it is preferable to use as a raw material eucalyptus, clove, allspice or the like, all of which belong to these genera, from a viewpoint of history of use in foods and safety. Especially, eucalyptus is preferable because eucalyptus has a very strong fructose absorption inhibitory activity, and an extract of eucalyptus leaves contains an affluent amount of the hydrolyzable tannin and contains Tellimagrandin I and II, Oenothein B, galloyl glucoses and the like in a large amount.

Among these, extraction using a solvent is performed under such conditions that the hydrolyzable tannin is eluted. For example, depending on the solvent used, extraction may be performed under the conditions of normal to increased pressure and a temperature from room temperature to the boiling point of the solvent for around 10 minutes to 1 week.

As the solvent used for extraction, solvents commonly used may be selected to use as appropriate according to the kind of plants and treatment processes. Examples of the solvents include water; organic solvents such as alcohols (for example, lower alcohols such as methanol and ethanol, or polyhydric alcohols such as ethylene glycol, propylene glycol, 1,3-butylene glycol and glycerin); ketones having relatively high polarity such as acetone; esters such as ethyl acetate, and the like. Among these, solvents combining methanol, ethanol or acetone with water are preferable. When the residue of an organic solvent is not preferable as in cases of being used as foods, it is especially preferable to use water, ethanol and aqueous ethanol. These solvents may be used solely or any two or more kinds thereof may be used in combination.

There are no specific limitations to the method for extracting the hydrolyzable tannin, and homogenizing extraction at room temperature, reflux extraction, supercritical fluid extraction and the like may be used.

For example, the following method may be used. An intact plant material or a dried plant material containing a large amount of the hydrolyzable tannin is pulverized. Then, 5 to 20-fold amount of an extraction solvent based on the total amount of the intact plant material or dried plant material is added to the pulverized plant. The mixture is allowed to stand under normal pressure at room temperature for around 1 week, or is extracted at around the boiling point of the extraction solvent for around 10 to 30 minutes. After that, the filtrate obtained by filtration is dried under reduced pressure or freeze-dried to obtain a plant extract.

The plant extract obtained as mentioned above may be used as it is, since the extract contains a large amount of the hydrolyzable tannin. Further, if needed, a purification treatment such as deodorization or decolorization may be additionally performed as long as the treatment does not affect the hydrolyzable tannin.

Any usual method may be selected to carry out the method for such purification treatments. For example, filtration, liquid-liquid extraction, ion-exchange resin, activated carbon column or the like may be used for adsorption, decolorization, purification and the like. Further, a purified product in a form of solution, paste, gel or powder may be obtained by freeze drying, a concentration treatment or the like.

There are no specific limitations to the form of the fructose absorption inhibitor of the present invention. For example, the fructose absorption inhibitor may be used in a unit dosage form which contains the predetermined amount of the hydrolyzable tannin in a desired formulation, or the extract or purified product obtained from plants using the above-described method may also be used as it is. When used in a unit dosage form, for example, it may be used as a composition containing the fructose absorption inhibitor and other components added as necessary.

Examples of such compositions include a composition containing the fructose absorption inhibitor and a suitable carrier (such as a carrier which is used for foods or drugs), and a composition containing the fructose absorption inhibitor and fructose.

There are no specific limitations to the formulation of the fructose absorption inhibitor. For example, any forms suitable for use as foods (food and drink), drugs, animal feed, additives for animal feed and the like may be used.

The content of the hydrolyzable tannin in the fructose absorption inhibitor of the present invention may be preferably 1 to 5000 mg, more preferably 10 to 3000 mg and especially preferably 50 to 1000 mg per unit dose of the fructose absorption inhibitor. When the content of the hydrolyzable tannin per unit dose of the fructose absorption inhibitor is less than 1 mg, there is a worry that the high fructose absorption inhibitory effect in the intestinal tracts is insufficient. On the other hand, when the content exceeds 5000 mg, there is a worry that an effect which meets the content of the hydrolyzable tannin cannot be obtained. In addition, intake of more hydrolyzable tannin than necessary can cause diarrhea depending on a person's constitution.

The unit dose refers to the predetermined amount calculated such that the fructose absorption inhibitory effect is exerted when the fructose absorption inhibitor of the present invention is taken in a form of tablets or other forms, in which form the amount is contained.

For providing a form of food, the hydrolyzable tannin is mixed with food materials to prepare a form of, for example, solid food, creamy or jam-like semi-liquid food, gel-like food, drink or the like. When used in such a form of food, especially use of fructose and/or the polysaccharides containing fructose in combination makes it possible to produce foods which are excellent in palatability and also can inhibit absorption of fructose.

When using the fructose absorption inhibitor in a form of food, various components commonly used for foods may be contained. Examples of such components include, glucose, maltose, sorbitol, stevioside, corn syrup, lactose, citric acid, tartaric acid, malic acid, succinic acid, lactic acid, L-ascorbic acid, dl-α-tocopherol, glycerin, propylene glycol, glycerin fatty acid ester, polyglycerin fatty acid ester, sucrose fatty acid ester, sorbitan fatty acid ester, propylene glycol fatty acid ester, gum arabic, carrageenan, casein, gelatin, pectin, agar, vitamin B complex, nicotinic acid amide, calcium pantothenate, amino acids, calcium salts, food colorings, flavors, preservatives and the like. These may be blended depending on the kind of foods as appropriate.

Specific examples of the foods include soft drink, juice, coffee, tea, liqueur, milk, whey beverage, lactic fermenting beverage, candy, chewing gum, chocolate, gummi, yoghurt, ice cream, pudding and the like. Addition of the extract or the fructose absorption inhibitor to foods is suitably carried out by adding the fructose absorption inhibitor so that the content of the hydrolyzable tannin is from 0.5 to 100 mg/g. In cases of supplements, there are no problems in safety and effect even if it is contained 90% by weight.

For use in a form of the pharmaceutical preparation, the hydrolyzable tannin is mixed with a pharmaceutically-acceptable common carrier to prepare a form of solid, semi-solid or liquid. Specific forms include, for example, oral administration agents such as tablets, capsules, pills, granules, powders, emulsions, suspensions, syrups and pellets, parenteral administration agents such as suppositories, and the like.

In formulating, conventionally used carriers such as surfactants, excipients, binders, disintegrant, lubricants, preservatives, stabilizers, buffers, suspensions may be used depending on the formulation. Preferably, examples of the carriers include, solid carriers such as starch, lactose, mannitol, carboxymethylcellulose, corn starch and inorganic salts; liquid carriers such as distilled water, physiological saline, an aqueous solution of glucose, alcohols (such as ethanol), propylene glycol and polyethylene glycol; oily carriers such as various animal and vegetable oils, white petrolatum, paraffin and wax; and the like.

Since the pharmaceutical preparation contains the hydrolyzable tannin effective for inhibiting fructose absorption as an active component, it has a fructose absorption inhibitory effect. Therefore, the pharmaceutical preparation is effective for prevention, improvement and treatment of various disorders and diseases caused by excess intake of fructose.

For example, the pharmaceutical preparation may be applied to use as preventive or therapeutic agents against diseases such as enhancement of oxidative stress in vivo, glycation of protein, calcium deposits in the kidney, hyperuricemia, ketosis, initiation of insulin resistance, initiation of cardiovascular kidney diseases and diabetic complication (such as diabetic kidney dysfunction and cataract, and such as necrosis of lower extremities) as well as hyperlipidemia, simple fatty liver and nonalcoholic fatty liver disease. In addition to that, the pharmaceutical preparation may also be applied to use as anti-obesity agents, fat accumulation inhibitors for visceral fat, subcutaneous fat and the like, anti-arteriosclerosis agents, thrombus prevention agents, hypotriglyceridemic agents, agents for lowering blood cholesterol levels and the like.

When producing animal feed which inhibits fructose absorption by using the hydrolyzable tannin in the present invention, one or two or more kinds of the extracts are mixed with various components used for animal feed for preparation.

In addition, the hydrolyzable tannin in the present invention may be used in a form of an additive for animal feed. In this case, the extract may be added to animal feed as it is. Alternatively, the additive for animal feed may be prepared to have a form of powder, granules, capsules, syrup, gel, liquid, solid or the like. Examples of the animal feed to which the additive for animal feed is added include the kinds of animal feed as mentioned above. The added amount may be the same level as the blended amount of the animal feed as mentioned above.

The animal feed may be added at any steps during production or after production.

### EXAMPLES

Hereinafter, the present invention will be described specifically with reference to examples, but the present invention is not limited to the following examples. Preparation of the extract and hydrolyzable tannin used in examples, evaluation of fructose absorption inhibitory activity, and handling of measured data were carried out by the following methods.

### <Method for Preparing Extract>

### (Crude Extract of Eucalyptus Leaves)

First of all, 5 kg of eucalyptus leaves was refluxed for 2 hours in 45 kg of 30% ethanol. Then, after cooled to room temperature, the resultant mixture was filtrated. The obtained filtrate was subjected to vacuum concentration and freeze drying to obtain a crude extract of eucalyptus leaves (yield: about 1 kg).

### (20% Ethanol-Eluted Fraction)

To a resin (“DIAION (registered trademark) HP20” manufactured by Mitsubishi Chemical Corporation), 100 g of the resultant crude extract of eucalyptus leaves was adsorbed. Elution was then carried out sequentially with 0 to 100% ethanol to obtain each eluate. And each fractionated eluate was subjected to vacuum concentration and freeze drying. Among these, the 20% ethanol-eluted fraction was found to have a strong fructose absorption inhibitory activity.

### (60% Methanol-Eluted Fraction)

Regarding the 20% ethanol-eluted fraction among the resultant each eluate, 5 g out of 16 g of yield was adsorbed to a resin (“TOYOPEARL (registered trademark) HW40 (f grade)” manufactured by TOSOH CORPORATION), and elution was carried out sequentially using 40 to 100% methanol as an eluent. High performance liquid chromatography was used for component analysis of each eluate. That is, while the solvent concentration was changed in the gradient mode, 5% acetic acid-acetonitrile (100%→0%) was flowed to a PAQ column (manufactured by NACALAI TESQUE, INC.) at 40° C., and component distribution of each eluted fraction was monitored with the measuring range of a photodiode array detector set at 270 to 350 nm. About 20 fractions were obtained by fractionation. Among these, the 60% methanol-eluted fraction was found to have a strong fructose absorption inhibitory activity.

### (Tellimagrandin I)

Regarding the 60% methanol-eluted fraction among each eluted fraction obtained by fractionating the 20% ethanol-eluted fraction, 80 mg out of 106 mg of yield was repeatedly subjected to HPLC fractionation by a PAQ column using the same eluent as mentioned above to obtain Tellimagrandin I (32 mg). Identification of the substance was confirmed by comparing the retention time of HPLC and various NMR data with those of the standard substance.

As other hydrolyzable tannins, those isolated and identified from various Myrtaceae plants such as eucalyptus leaves were used.

### <Evaluation Method of Fructose Absorption Inhibitory Activity in the Intestinal Tracts>

Evaluation of fructose absorption inhibitory activity was carried out using the human colon carcinoma cell line, Caco-2 (manufactured by Dainippon Sumitomo Pharma Co., Ltd.) utilized for model experiments of the small intestinal mucosa. Medium was prepared for use by adding FCS (manufactured by BIOWEST) and NEAA (manufactured by SIGMA) to DMEM medium (manufactured by SIGMA) such that the content of FCS is 10% and that of NEAA is 1%.

Caco-2 cells were seeded in a 6-well Transwell Insert (internal area: 4.2 cm²) and subcultured for around 3 weeks, while DMEM medium was replaced once every 3 to 4 days, until the Caco-2 cell density became around 2×10⁵ cells/insert. The TEER value was measured before experiments and the state of Caco-2 cells was confirmed. Each sample illustrated in Table 1 was dissolved in 10% dimethyl sulfoxide (DMSO).

Medium in the 6-well Transwell Insert was removed. After both of the inside and outside of the insert were washed with a phosphate buffer solution (pH 7.2) (PBS), D-PBS (manufactured by GIBCO) which did not contain saccharides and serum was added to the insert. After 30 min of incubation at 37° C. under 5% CO₂, the TEER value was measured. A sample solution of each sample illustrated in Table 1 was added to the insert and each mixture was preincubated for 5 min. A fructose solution was added such that the final concentration of fructose is 50 mM and each mixture was incubated at 37° C. under 5% CO₂ for 3 h. The TEER values were measured. Only for ones whose TEER values did not reduce after the culture, a permeated solution outside the insert was collected and stored at −80° C. The measurement was carried out three times for each sample illustrated in Table 1.

The fructose concentrations of the permeated solutions were measured by an enzymatic method using D-fructose dehydrogenase (derived from Gluconobac sp.) (The concentration of each reagent represents the final concentration, respectively.). That is, to a mixed solution containing 100 mM PBS (pH 6.0), 1% Triton X-100, 0.2 mM WST-1, 8 μM 1-methoxy PMS and 10 U fructose dehydrogenase (manufactured by TOYOBO CO., LTD.), the permeated solution was added. The resulting mixture was reacted at 30° C. for 3 h, followed by measurement of the absorbance at 438 nm.

The fructose absorption inhibitory rate of each compound was determined using the following formula (1). Results are illustrated in Table 1. As control, measurement was also carried out for gallic acid, ellagic acid, quercetin, (+)-catechin, (−)-epicatechin and (−)-epigallocatechin gallate.

(Permeated amount of fructose for blank (10% DMSO)−Permeated amount of fructose for sample)/Permeated amount of fructose for blank×100 (I)

TABLE 1

| Sample | | | Dosage | Inhibitory |
|---|---|---|---|---|
| No. *1 | | Name of Samples | (μg/ml) | Rate (%) |
| | 1 | Tellimagrandin I | 5 | 49 |
| | 2 | Tellimagrandin II | 5 | 52 |
| | 3 | Strictinin | 5 | 43 |
| | 4 | Casuarictin | 5 | 46 |
| | 5 | 1,3-di-O-galloyl-4,6-HHDP-β-D-glucose | 5 | 57 |
| | 6 | Eugeniflorin D₂ | 5 | 53 |
| | 7 | Oenothein B | 5 | 63 |
| | 8 | 1,2,3,4,6-pentagalloyl glucose | 5 | 53 |
| | 9 | 1,2,3,6-tetragalloyl glucose | 5 | 50 |
| | 10 | 1,2,3-trigalloyl glucose | 5 | 61 |
| * | 11 | Gallic acid | 50 | 19 |
| * | 12 | Ellagic acid | 50 | 22 |
| * | 13 | Quercetin | 50 | 23 |
| * | 14 | (+)-catechin | 50 | <10 |
| * | 15 | (−)-epicatechin | 50 | <10 |
| * | 16 | (−)-epigallocatechin gallate | 5 | 28 |

*1: Comparative Examples are indicated with “*”.

As illustrated in Table 1, the hydrolyzable tannins of samples of No. 1 to 10 had 43% or more of the inhibitory rate, even though the dosage was only 5 μg/mL. That illustrates the hydrolyzable tannins of samples of No. 1 to 10 had a strong fructose absorption inhibitory activity. From this result, it is expected that the hydrolyzable tannin inhibits absorption of fructose into the body in the small intestine and suppresses obesity and the like caused by excess intake of fructose. Examples illustrate that the dosage of 5 μg/mL of the hydrolyzable tannin is effective in the absorption model experiments using the intestinal epithelial cell, Caco-2 (surface area: 4.2 cm²). However, in mammals including humans, intestinal epithelial cells to absorb fructose exist in a far greater number than those in this absorption model experiments, and its surface area is estimated to reach 200 m² in humans. Therefore, clinically, the effective content of the hydrolyzable tannin in the present invention is considered to be at least 1 mg, preferably 10 mg or more, and more preferably 50 mg or more.

On the other hand, in the case where the crude extract of eucalyptus leaves (corresponding to the extract in Patent Document 1) was used, the dosage of 1000 μg/mL led to the inhibitory rate of 65% and the dosage of 100 μg/mL led to the inhibitory rate of less than 20%. Thus, in order to exert fructose absorption inhibitory activity by using the crude extract of eucalyptus leaves as it is, the dosage of 1000 μg/mL is needed.

The above-described Examples do not limit the present invention and are of course applicable within a scope without departing from the spirits of the present invention. The above-described Examples explain only one component obtained from eucalyptus leaves (Tellimagrandin I). However, not only eucalyptus but also other plants containing the hydrolyzable tannin may be used, and they may be partly purified for use as well. Further, a mixture of the hydrolyzable tannins may also be used.

1. A fructose absorption inhibitor comprising a hydrolyzable tannin as an active component.

2. The fructose absorption inhibitor according to claim 1, wherein the hydrolyzable tannin is a compound having an ester bond composed of a hydroxyl group in glucose and one or more of a gallic acid derivative or an ellagic acid derivative.

3. The fructose absorption inhibitor according to claim 2, wherein the gallic acid derivative is a compound represented by the general formula (1): 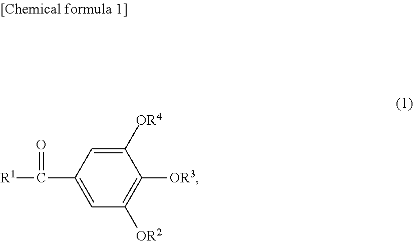
wherein R¹ represents a hydroxy group or an alkoxy group, and R², R³ and R⁴, which are the same or different, each represent a hydrogen atom, an alkyl group or an acyl group.

4. The fructose absorption inhibitor according to claim 2, wherein the ellagic acid derivative is a compound represented by the general formula (2), a compound having a hexahydroxydiphenoyl group derived from an oxide of an ellagic acid, or a compound having a valoneoyl group in which a gallic acid is added to a hexahydroxydiphenoyl group: 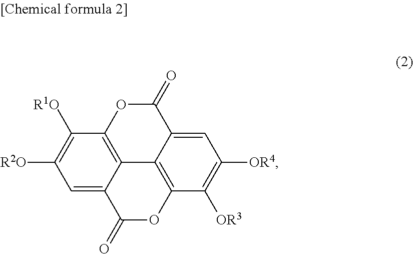
wherein R¹, R², R³ and R⁴, which are the same or different, each represent a hydrogen atom, an alkyl group or an acyl group.

5. The fructose absorption inhibitor according to claim 1, wherein the hydrolyzable tannin is ellagitannin having an ester bond composed of hydroxy groups at the 2- and 3-positions or the 4- and 6-positions or at the both positions in glucose and at least one hexahydroxydiphenoyl group or valoneoyl group.

6. The fructose absorption inhibitor according to claim 1, wherein the hydrolyzable tannin is gallotannin having an ester bond composed of at least three hydroxy groups in glucose and gallic acids.

7. The fructose absorption inhibitor according to claim 1, which has a form of food or drug.

8. The fructose absorption inhibitor according to claim 1, which has a form of animal feed or an additive for animal feed.

9. The fructose absorption inhibitor according to claim 1, wherein the hydrolyzable tannin is at least one selected from the group consisting of Strictinin, Casuarictin, Oenothein B, 1,2,3-trigalloyl glucose and Eugeniflorin D₂.
